(19) United States
(12) Patent Application Publication
(10) Pub. No.: US 2007/0293742 A1
(43) Pub. Date: Dec. 20, 2007

(54) Medical System And A Method Of Controlling The System For Use By A Patient For Medical Self Treatment

(75) Inventors: Jan Henning Simonsen, Struer (DK); Jens Ulrik Poulsen, Virum (DE); Kent Halfdan Rokkjaer, Holstebro (DK); Lars Hofmann Christensen, Jyllinge (DK); Soren Aasmul, Holte (DK); Steffen Lav, Bronshoj (DK)

Correspondence Address: NOVO NORDISK, INC.;PATENT DEPARTMENT, 100 COLLEGE ROAD WEST, PRINCETON, NJ, 08540, US

(73) Assignee: Novo Nordisk A/S, Bagsvaerd (DK)

(21) Appl. No.: 11/846,028

(22) Filed: Aug. 28, 2007

Related U.S. Application Data
(63) Continuation of application No. 10/342,150, filed on Jan. 14, 2003.
(63) Continuation of application No. 09/450,193, filed on Nov. 29, 1999, now Pat. No. 6,540,672.

(30) Foreign Application Priority Data
Nov. 30, 1998 (DK) PA 1998 01578

(51) Int. Cl. A61B 5/00

(52) U.S. Cl. 600/316

(57) ABSTRACT

This invention relates to an system for self treatment. The system consists of several portable modules where one of the modules is designated as a master module. The master module controls, supervises and monitors all the mutual information and data exchange between itself and the rest of the modules. The modules can e.g. consist of a BGM, a doser and a storage container. The modules may be able to generate and store data which is transmitted to the master module if it is within range. If the master module is not within range, the data is kept locally in the module until the master module is available. The master module can send the data to an external unit like a computer or database for further processing. A physician or an expert care-team can access the data in the database and give guidance to the patient on the basis of these processed data. This processing could also be done automatically by utilization of an expert system.

### CROSS REFERENCE TO RELATED APPLICATIONS

This application is a continuation of application Ser. No. 10/342,150 filed on Jan. 14, 2003 and claims priority under 35 U.S.C. 119 of provisional application No. 60/111,721 filed Dec. 9, 1998 and Danish Application No. PA 1998 01578 filed Nov. 30, 1998. The benefit of U.S. non provisional application Ser. No. 09/450,193 filed Nov. 29, 1999 is claimed under 35 U.S.C. 120; the contents of all are fully incorporated herein by reference.

This invention comprises a medical system and a method of controlling the system for use by a patient for medical self treatment.

For a number of years it has been possible to purchase various devices for the treatment of diabetes, e.g. for injecting insulin, for measuring blood sugar (such a device is referred to as BGM in the following), for withdrawing blood samples, and other accessories, the purpose of which is to enable the patient to nurse his disease discretely and with a high standard of safety.

Many diabetic patients are elderly people who can easily get insecure with respect to the medical equipment. It is very reassuring and therefore also very important that the user can have feedback from the system which confirms to the user that everything is OK right from the technical function of the system to the patient's physiological condition. This stretches out a psychological safety net under the patient, which contributes to improving the quality of life of patients having a disease such as diabetes.

Also many young people need to assure themselves that the equipment is in order, i.e. calibrated, powered, updated and otherwise ready to be operated.

One way of ensuring that you have all the things needed ready at hand is to build several of the necessary devices together into a single integral unit, see e.g. U.S. Pat. No. 5,536,249. This is not an ideal solution since such a multi-functional device is usually quite complex both with respect to manufacture and use. People need to be familiar, secure and confident with the use of a device for self-treatment which such an integral multi-functional does not provide.

Another drawback of integrating several functions in one apparatus is that owing to the commercial outlets the manufacturer never integrates all possibilities, but just the most important ones, in order for it to be relevant to a sufficiently large group of users. The functions which are thus not integrated must be provided by means of separate apparatuses typically of different makes, which can easily create uncertainty as to whether the apparatuses work correctly together.

According to the invention the individual devices may be arranged for various respective functions relevant to the treatment of e.g. diabetes, such as: a lancet device, a body fluid analyser, one or more drug administration apparatuses for administering a predetermined dose of medication to the patient. Further, there is a number of other aids which the diabetic patient uses, e.g. test strips for the blood analyser, needles, napkins for wiping off blood, extra insulin carpoule, glucose tablets, waste containers, etc.

The object of the invention is to provide a method for effective monitoring of electronic data relevant to a plurality of apparatuses which are used by a patient for self-treatment of a disease, so that a greater level of safety, both functionally and emotionally, and an effective feedback to the patient are obtained.

This is achieved according to the invention in that the individual apparatuses are provided with electronic communications equipment so that the apparatuses—when in a state of mutual communication—frequently exchange information between them. Hereby a greater functional safety can be achieved and the total data capacity of the system can be increased, so that the feedback possibilities, e.g. of the system checking that every apparatus is OK and set up properly and of the patient be given a number of possible choices to choose from in a given situation, are increased.

More particularly, the invention relates to a method of controlling data information between a plurality of portable apparatuses for use by a patient for medical self treatment, the treatment including a first operation and at least a second operation, said portable apparatuses comprising a first apparatus for performing the first operation, and at least a second apparatus for performing the second operation, wherein each apparatus belonging to the medical self treatment has means for one or more of the following: storing, transmitting, receiving and displaying information, and an attempted data communication between said apparatuses is initiated on request.

The request can e.g. be initiated by a timer or other external events such as the patient performing an action.

The invention provides the further effect that a patient need not bring along a large apparatus technically complicated in use in order to treat his disease, but that the apparatus may be divided into several smaller and simpler units capable of communicating mutually. The individual units may optionally be adapted to be interconnected mechanically, as disclosed in Danish Patent Application No. PA199800714.

According to the invention, all apparatuses need not be active for communication to be established between some of the apparatuses. This requires that all the apparatuses are adapted to a specific communications protocol, there being several options in this respect. For example, one of the units may be provided with program information of highest priority with respect to the control and monitoring of data communication between the individual apparatuses. The unit of highest priority may very well be turned off, because the apparatuses may be adapted to communicate directly and perform storage of information, which is subsequently transmitted to the unit of highest priority when this is again in communication with another of the apparatuses.

Alternatively, other communications protocols may be implemented such as:

A protocol where a number of potential master modules (unit of highest priority) is predefined. These predefined master modules are given a hierarchical priority and the master module with the highest priority among the activated and present master modules becomes the functional master module. This master module polls the other activated and present apparatuses for information.

A protocol where all the apparatuses can request information/data exchange with one or more of the other possible apparatuses (present and activated) on the basis of an event (e.g. timer based, user operation, etc.). This is done by sending an interrupt request (IRQ) to the other apparatuses which they can act upon according to their situation. With this protocol there is not a designated master module and every apparatus can communicate/exchange data with one or more of the rest of the apparatuses.

A protocol for a self-organizing network where every apparatus retransmits all the received information until the apparatus or apparatuses which the information was meant for receive it. In this way every apparatus functions as a relay station and a temporary store of transmitted information. This structure is especially useful when the configuration of the network is not known or when the configuration of the network changes in an unpredictable manner. Another feature of a network of this kind is that a maximal number of redundant transmission paths with a buffer are created so that the system can transmit information to apparatuses which were not available when the information was transmitted.

A protocol where all the apparatuses transmits their information without any supervision of any kind. The apparatuses themselves have to decide what information is relevant for them.

The unit of highest priority may be adapted to communicate with a larger communication center which may contain a patient database. Such further use of the invention is known e.g. from U.S. Pat. No. 5,204,670, which, however, cannot offer the patient the flexible and safe use of a set of different apparatuses according to the invention which together are used in the treatment of a disease.

The apparatuses according to the invention communicate information such as: amount of medication, type of medication, the concentration of relevant substances in the body e.g. body fluid level/concentration, time stamp, amount of food (e.g. amount or units of carbohydrate), measurement of physical activity, notification (e.g. alert and warning) to the patient, body characteristics (e.g. weight, blood pressure etc.) and inventory logistics. This ensures that relevant information for e.g. a drug administration system like a doser, i.e. number of units of insulin, insulin type and time and date for administering, can automatically be stored, displayed, received and transmitted to and from all the relevant apparatuses. The doser could also receive information regarding a predetermined number of units of insulin to be administered and automatically set the amount of medication to be administered by electromechanical means. In this way elderly and handicapped people do not have to set the relevant amount of medication themselves but just activate the doser.

It is especially useful to transmit the data from all apparatuses to the apparatus containing the highest priority program for safe keeping, calibration and updating of data and possible transmission to e.g. an external unit like a PC or database for further data acquisition, storage and processing.

In this way the patient, a physician or an expert care-team can obtain the behavior over time of the patient, and a check for compliance to a diet or treatment given to the patient by a physician or an expert care-team can be made. This could also be done automatically.

Additionally, it is also possible for the patient to manually input information about the treatment. This information may be historic information as well as information about a future scheme (behavioral pattern) e.g. planned physical exercise, administering of insulin, intake of food and other medications. This information may be collected and thus serve as an electronic diabetes diary or may be used to notify the patient through the receiving means as to whether the planned actions are dangerous or not.

It is evident that since the apparatuses are to be carried by the patient, there is a potential lack of space for an advanced input device e.g. a keyboard. Therefore, information which cannot be input on a standardized form e.g. personal comments on the treatment is typed into the apparatus by the patient using a simple input device once and can subsequently be chosen from a list if needed again.

The patient can further receive recommended amounts of medication, exercise, food, etc. from a physician, an expert-team or automatically.

The invention also relates to an apparatus for a medical apparatus for use by a patient for medical self treatment, the treatment including a first operation and at least a second operation, the apparatus comprising a first apparatus for performing the first operation and a second apparatus for performing the second operation, wherein each apparatus comprises means for storing and/or displaying information, and comprising means for transmitting and receiving information so that each apparatus is able to exchange data with any of the other apparatuses belonging to the self treatment.

For a BGM according to an embodiment of the invention the relevant information could be the time and date for measurement, measured level/concentration of blood glucose which could be stored or transmitted to another apparatus.

For a doser according to an embodiment of the invention the relevant information could be the type of medication (e.g. long acting or short acting insulin), number of units of insulin to be administered and the time and date of the administering. This information could both be set manually by the patient or remotely by a physician, an expert care-team or automatically.

For a storage container according to an embodiment of the invention the relevant information could be to keep track of the contents of the container so that every time an object (e.g. carpoule, needle, etc.) is used, the storage container will update the inventory list. This list could be transferred to an unit of highest priority immediately or later, which could in turn update the patient's total holdings of objects, so that the system could notify the patient when he should order a new stock of objects. The ordering could also be done automatically by the system if the inventory list is transferred to an external unit, which greatly improves the confidence, comfort and safety of the patient.

In the following a preferred embodiment according to the invention is described in detail. This particular embodiment is meant as one example only of the invention and should not as such limit the scope of protection.

In the preferred embodiment a specific simple communication protocol has been chosen to simplify the explanation of the invention. In the chosen protocol a predefined apparatus is chosen as the unit of highest priority (master module) which controls, coordinates and monitors the mutual data communication between all the apparatuses including itself. The master module collects or mirrors all the data stored in the other apparatuses. This collected or mirrored data can then be redistributed to any of the other apparatuses or an external unit (e.g. a personal computer or database system) for later retrieval and/or processing.

According to the invention the portable system can operate even if the master module is not present, since all the relevant apparatuses comprise internal storage means, so that they can store the relevant information when it is obtained and transmit it when they can reach the master module once again.

Preferably the information obtained is kept in the apparatuses so that the patient on request always can be presented with the latest measurements and/or information obtained or received.

A person skilled in the art could easily implement other communication protocols such as the ones described above.

In this embodiment a cap unit for a doser has been chosen as the master module but any apparatus could have been chosen just as easily. Preferably, the master module should be the apparatus that the patient carries most often.

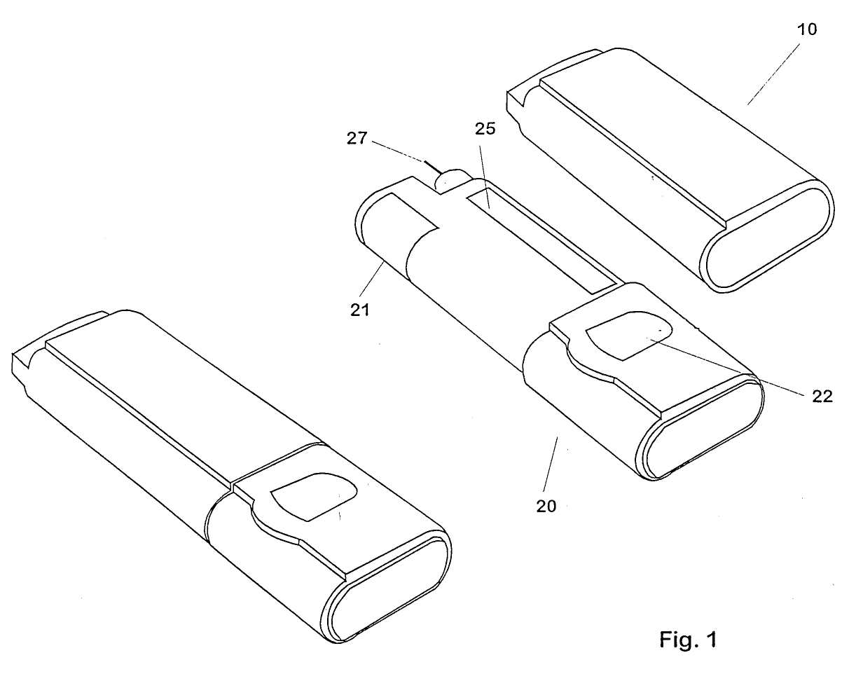

The invention will now be explained in detail with reference to the FIGS. 1-9, in which

FIG. 1 shows a prior art doser with a conventional cap;

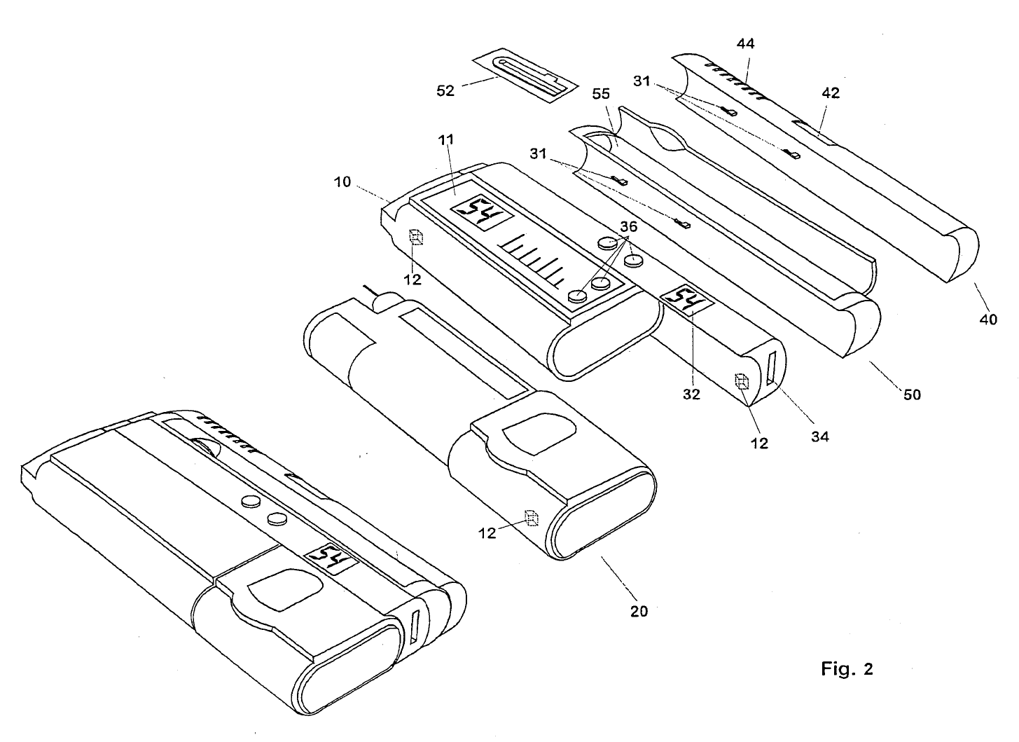

FIG. 2 shows a doser and a cap with a BGM, a lancet device and a container for test strips attached;

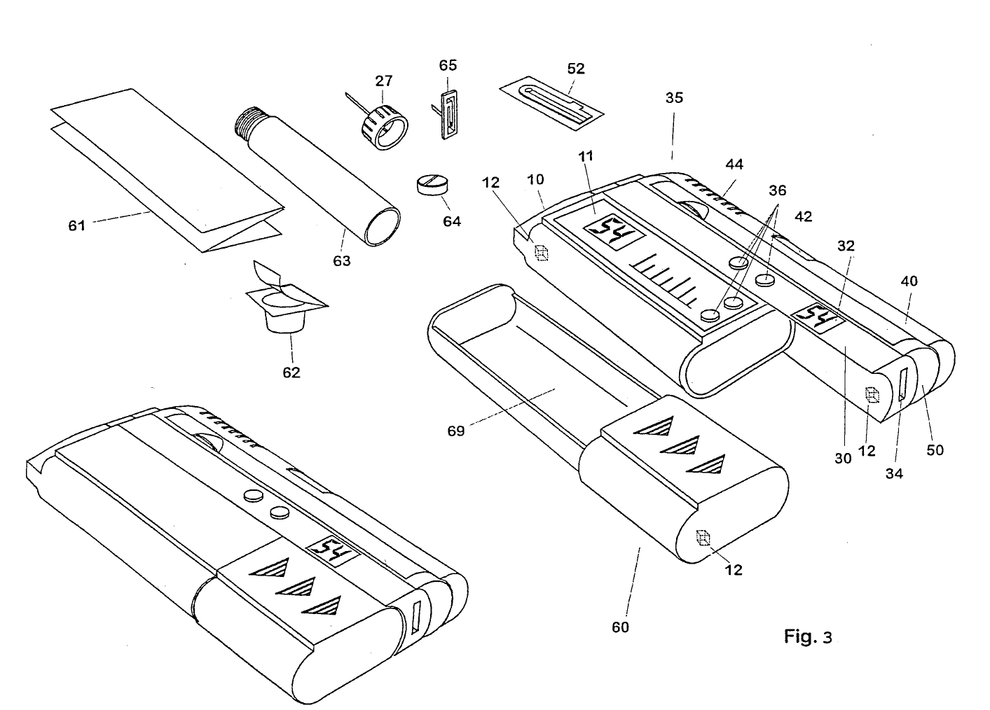

FIG. 3 shows a cap with a BGM, a lancet device, a test strip container attached and an additional container together with useful/needed extras;

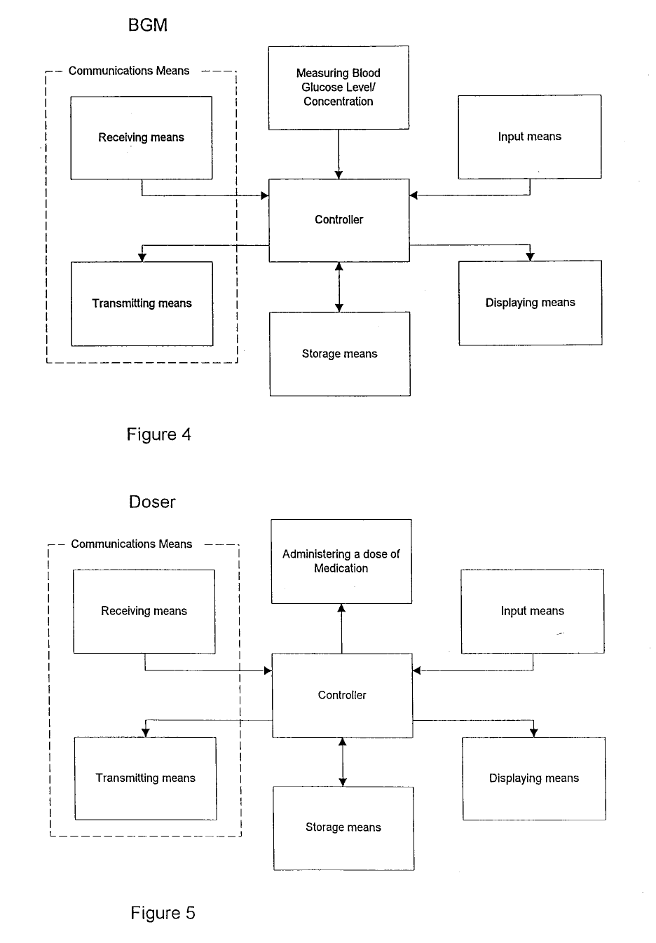

FIG. 4 shows a schematic functional diagram of a BGM according to an embodiment of the invention;

FIG. 5 shows a schematic functional diagram of a doser according to an embodiment of the invention;

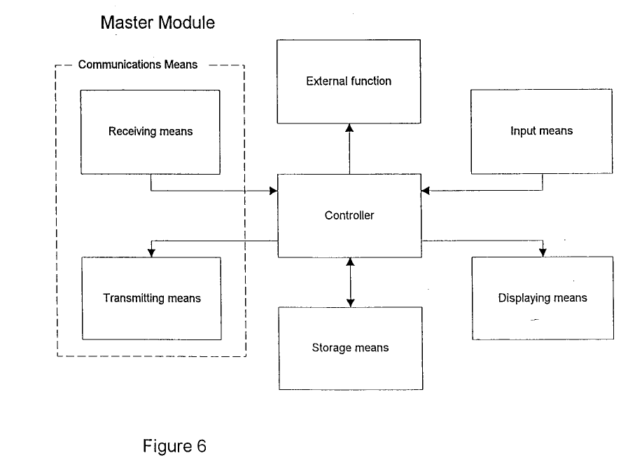

FIG. 6 shows a schematic functional diagram of a unit of highest priority according to an embodiment of the invention;

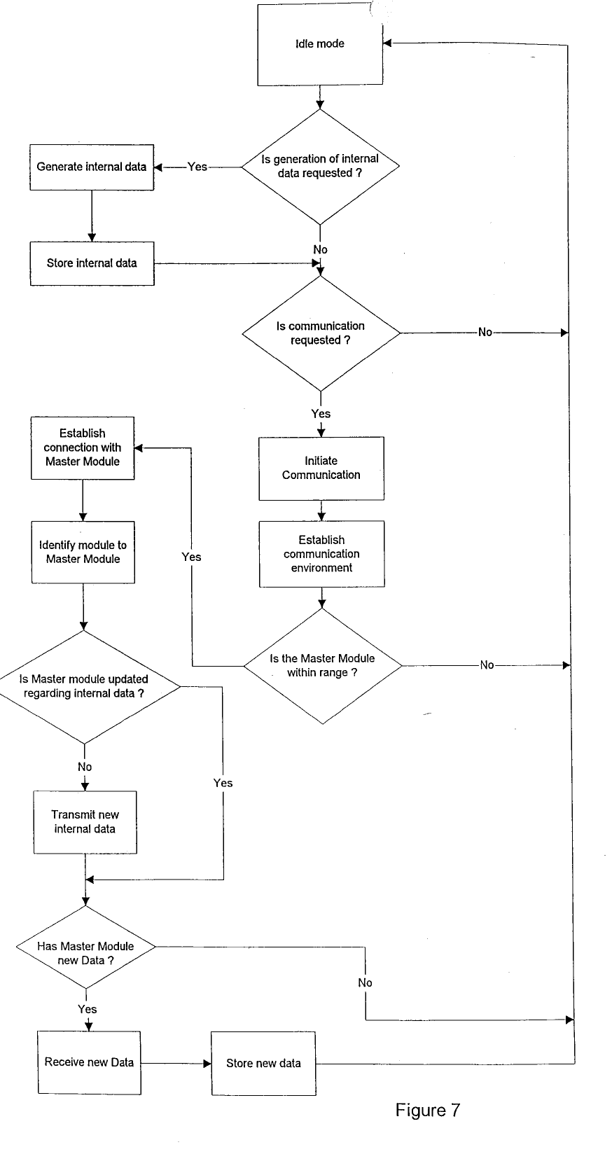

FIG. 7 shows a flowchart illustrating an apparatus generating new data (e.g. a BGM) and how the apparatus behaves with respect to data generation and communication;

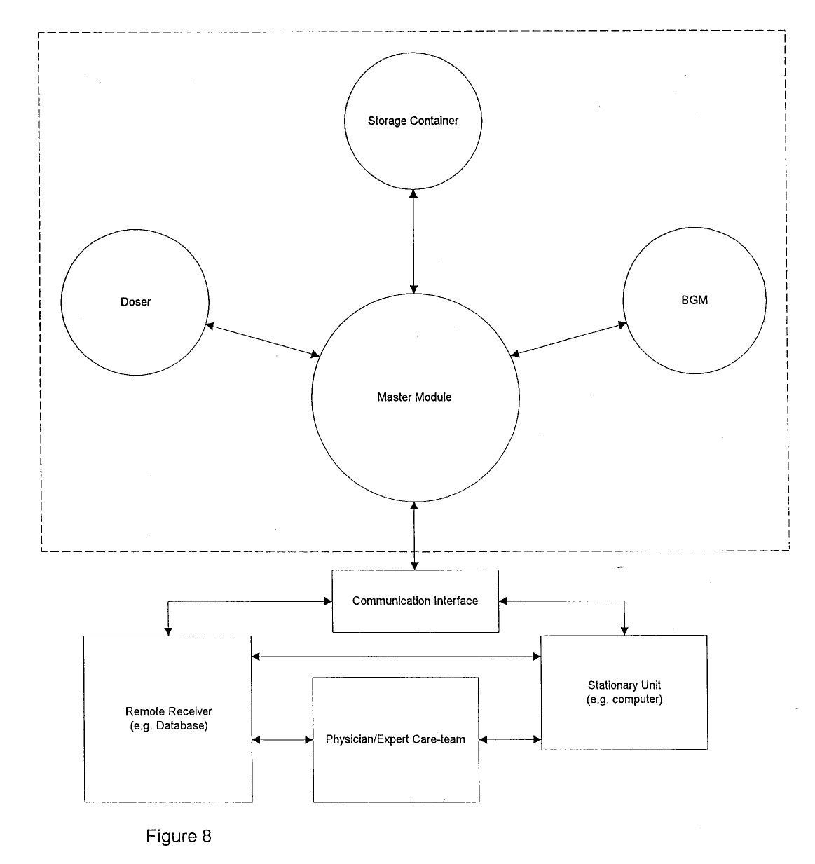

FIG. 8 illustrates the general concept according to an embodiment of the invention with respect to communication;

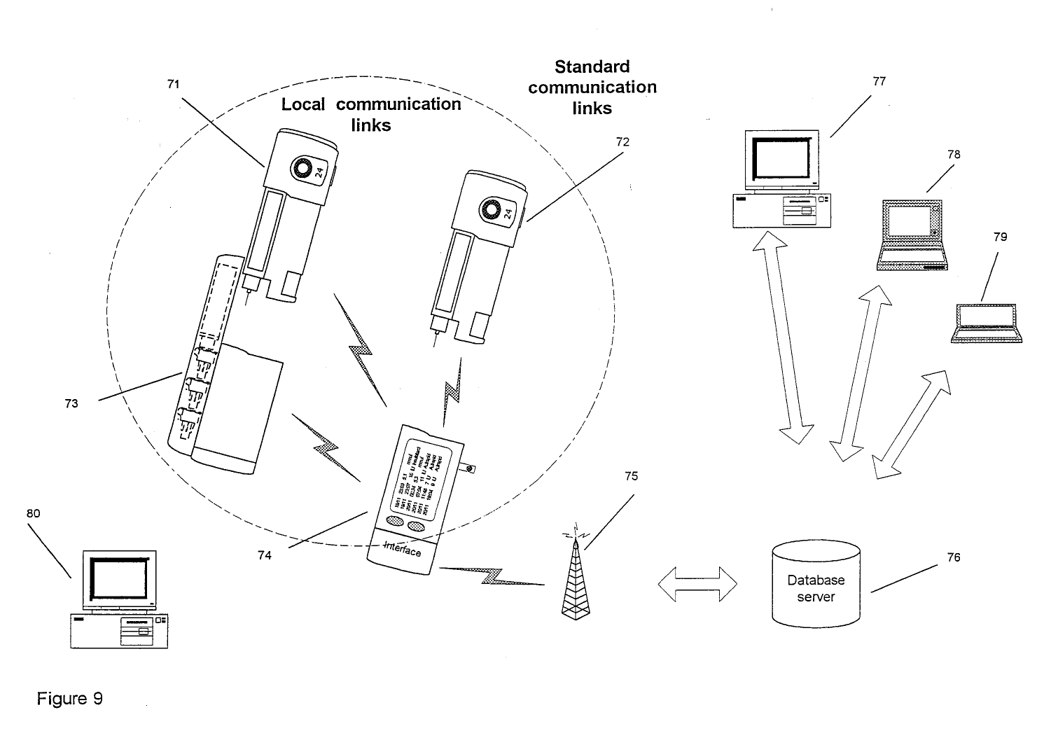

FIG. 9 illustrates two dosers and their communication paths.

FIG. 1 shows a prior art doser 20 and a cap 10. The doser 20 comprises a turning wheel 21 for adjusting, either electronically or manually, the level/amount of medication to be administered, and a display 22 that shows the currently selected amount of medication to be administered. The doser 20 has processing means and storage facilities, like a CPU and RAM, for storing data, like the time, date and amount of medication of the last couple of administrations. This information can be shown in the display 22 at request. The doser 20 further comprises a carpoule (not shown) that contains the medication, and is fitted with a needle 27 through which the medication is administered. The doser 20 has a transparent window 25 so that the amount of medication left in the carpoule can readily be identified. The cap 10 can be fitted to the doser 20 so that one single compact unit and protection of the doser 20, needle 27, etc. are obtained.

FIG. 2 shows a doser 20 with a cap 10 where the cap 10 functions as the master module. The doser 20 corresponds to the doser 20 shown in FIG. 1 but with the additional feature of having transmitting and receiving means 12. This enables the doser 20 to transmit the stored data, i.e. the time, date, amount and type of medication, to the master module 10 for storage and presentation there via the master modules receiving means 12. Information of the last couple of administrations (time, date, type and amount of medication) can then easily be viewed on the display 11 on the master module. If the master module 10 is not present or active, the doser 20 will just store the information locally until the master module 10 becomes available and the patient will be able to view the information on the doser 20.

The doser 20 can also receive information via the receiving means 12 from the master module 10. This information could for instance be a predetermined amount of medication dictated remotely by a physician, an expert care-team or automatically. The received information is then used to automatically set the correct amount of medication to be administered so that the patient does not have to worry about that aspect, which is a great advantage especially if the patient is elderly or handicapped.

Also shown is a BGM 30 which has means 34 for inserting test strips 52 containing a sample of blood, for analysis by the BGM 30 by operating the buttons 36. The result of the analysis is stored and either shown in the display 32 or transmitted to the master module 10 via the transmitting means 12 for storage and presentation on the larger display 11. The patient can at the same time be presented with the last couple of results over a time period.

A test strip container 50 is provided for the safe keeping/storing of test strips 52 in the space 55 and can be added/attached through locking means 31. With this addition, a test strip 52 will always be available.

Further shown is a lancet device 40 removably attached to the BGM 30 by the locking means 31. This lancet device 40 is used by first loading the lancet device through the grip 44 and then pressing the button 42, which releases the lancet, piercing the skin, so that a blood sample can be obtained. With this inclusion, the lancet device 40 is always at hand. This has the advantage that a lancet device 40 is always available, for taking a blood sample and applying it to a test strip 52. The test strip 52 can then be inserted via the means 34 into the BGM 30, which will start analysing the blood sample and, after completion of the analysis, will show the result in the display 32. It is very useful to have the BGM 30 and the lancet device 40 attached together in one compact unit, since a BGM 30 would not normally be used without the lancet device 40, thereby avoiding the fuss and uncertainty of using multiple devices of perhaps different makes. On the other hand, if the user already has a lancet device and is accustomed to and familiar with the use of this particular lancet device, he can still use this original lancet device and just use the remaining items, which will be a compact set consisting of a doser 20 and a BGM 30; The cost will be reduced hereby.

FIG. 3 shows the same units as are shown in FIG. 2, but instead of a doser 20, there is now provided a container unit 60 with a relative large space 69 for storing the items needed everyday for self-treatment. For a diabetic, e.g. such items could be a napkin 61 for wiping excessive blood after a sample has been taken, a waste container 62 for receiving used items, an extra carpoule 63 which could contain another type of insulin, spare needles 27 for the doser, spare lancets 65 for the lancet device 40, some glucose in the form of glucose tablets 64, etc. In some situations and in certain forms of diabetes, the injection of insulin may be replaced by administration of pills which may be stored in the container, which thus replaces the doser described previously. All these items, or the most relevant ones for a given situation, could be held in the container space 69 for easy retrieval, when needed.

The container unit 60 is provided with transmitting, receiving and storage means 12. These means are used to communicate an inventory list to the master module 10 on which the user can view and update the inventory list via the buttons 36.

This list could be transferred to an external unit (e.g. computer, laptop, palmtop, etc.) immediately or later, which could update a list of the patient's total holdings of objects, so that the system could notify the patient when he should order a new stock of objects. The ordering could also be done automatically by the system. In this way the patient will not have to be concerned whether he has all the necessary objects for a future span of time or not, which greatly improves the confidence and safety of the patient.

FIG. 4 shows a schematic functional diagram of a BGM according to an embodiment of the invention. The BGM consists of the following functional blocks: ‘Controller’, ‘Receiving means’, ‘Transmitting means’, ‘Storage means’, ‘Displaying means’, ‘Input means’ and ‘Measuring Blood Glucose Level/Concentration’.

The central block is the functional block ‘Controller’ which coordinates, monitors and controls the tasks of all the other functional blocks. The ‘Receiving means’ and ‘Transmitting means’ is responsible for receiving and transmitting of information data, respectively. The block ‘Measuring Blood Glucose Level/Concentration’ performs the measurement of the blood glucose level/concentration on e.g. a test strip, containing a blood sample. The ‘Displaying means’ can display relevant information to the patient e.g. the result of a measurement and a time stamp containing the time and date of the measurement. The result of the measurement can be stored in the ‘Storage means’ for later retrieval and further be sent to another apparatus (e.g. the master module) through the ‘Transmitting means’. All these tasks take place under the supervision and coordination of the ‘Controller’ block.

The BGM according to an embodiment of the invention could thus be operated in the following way. When a request for a measurement of the blood glucose level/concentration is made either by the patient through the ‘Input means’ or by another apparatus through the ‘Receiving means’, the controller receives the request and activates the ‘Measuring Blood Glucose Level/Concentration’ block, which initiates and performs the measurement of the blood glucose level when the patient inserts a test strip with a sample of blood into a slot on the apparatus. Previously a calibration of the measurement equipment could be made by insertion of a calibration test-strip. The result and a time stamp of the measurement are then transferred to the storage means, and the controller can send the result via the transmitting means to another device e.g. the master module if it is within range.

All these functional blocks could be implemented by prior art/standard components. The block labeled ‘Controller’ could e.g. be implemented by any type of CPU, micro processor, micro controller, EEPROM or ROM containing software, firmware, etc. The functional block ‘Storage means’ could be standard RAM.

The BGM is only an example of an apparatus that could be used according to this invention. Any other body fluid analyser e.g. lipid monitor or the like could be used.

FIG. 5 shows a schematic functional diagram of a doser according to an embodiment of the invention. The doser consists of the following functional blocks: ‘Controller’, ‘Receiving means’, ‘Transmitting means’, ‘Storage means’, ‘Displaying means’, ‘Input means’ and ‘Administering a dose of Medication’. These functional blocks correspond to the blocks previously described for the BGM in FIG. 4, except for the block ‘Administering a dose of Medication’, and will therefore not be explained once more.

The functional block ‘Administering a dose of Medication’ administers a dose of medication e.g. insulin. The amount of medication could be set by the patient through the ‘Input means’ or be set electromechanically by the ‘Controller’ block according to information received via the ‘Receiving means’. This information could be prescribed by a physician, by an expert care-team or automatically, so that elderly or handicapped people would only have to activate the doser through the input means to be administered a dose of medication. After the activation of the doser, information e.g. type of medication (e.g. long acting or short acting insulin), amount of medication and the corresponding time stamp (date and time) is stored in the ‘Storage means’ and transmitted to an apparatus (preferably the master module) if it is within range.

Other devices than an insulin doser could be used in accordance with the invention. These could e.g. be devices that administer growth hormones, etc. One could also have an device that obtains information of orally obtained medication like OHA (Oral Hyperglychemical Agent). This would, however, require the user to manually input the type and amount of medication, which could be done by choosing icons, selecting an object in a predetermined list or typing the information by alphanumeric keys. Preferably, a predetermined list would require the user to just enter (e.g. by icons or alphanumeric keys) the relevant text once and then later just present the user with the already entered text and only ask for the amount and type (which could also be pre-entered in the same fashion).

FIG. 6 shows a schematic functional diagram of a master module according to an embodiment of the invention. The master module consists of the following functional blocks: ‘Controller’, ‘Receiving means’, ‘Transmitting means’, ‘Storage means’, ‘Displaying means’, ‘Input means’ and ‘External function’. These functional blocks correspond to the blocks previously described with reference to FIGS. 4 and 5, except for the block ‘External function’, and will therefore not be explained once more.

The master module is the module responsible for the coordination, supervision and control of the information and data exchange between itself and all the other present and activated apparatuses. These apparatuses identify themselves to the master module when they are within range so that the master module always knows which apparatuses are present and activated. The master module also receives and stores all the information and data generated in the individual apparatuses for later retrieval and/or transmission to an external unit (e.g. computer or database) for further storage and processing. The relevant information can be displayed on the larger display of the master module and be acted upon by the patient.

Some of the tasks of the master module could be implemented in the external unit and vice versa.

The master module could be any of the apparatuses as represented by the functional block ‘External function’ in FIG. 6, but is in this embodiment the cap unit 10 shown in FIGS. 2-3, and has as such no external function. Other functions may readily be implemented in this block.

FIG. 7 shows a flowchart illustrating an apparatus generating new data (e.g. a BGM) and how the apparatus behaves with respect to data generation and communication.

In idle mode the apparatus determines whether or not data generation is requested. If this is the case (e.g. if the user has inserted a blood glucose measuring strip into the apparatus), the data generation block assumes priority and completes the procedures associated with the data generation (e.g. measurement of the blood glucose concentration). After completion of the data generation the data is stored in the internal memory of the apparatus.

After completion of the data generation or after determination that data generation was not requested, the apparatus determines whether or not communication is requested—either by the apparatus itself (several criteria can issue the communication request e.g. a timing event, a user interface event, etc.) or by an apparatus different from the apparatus itself (e.g. a request from the master module). If communication is not requested, the apparatus resumes its idle mode. If communication is requested, the apparatus sends out a request for the other apparatuses within its range to identify themselves to the apparatus—enabling it to establish the present communication environment. Based on the established communication environment the apparatus identifies whether or not the master module is within range of the apparatus. If the master module is not within range of the apparatus, the communication is terminated and the apparatus returns to its idle mode. If however, the master module is within range of the apparatus, the apparatus sets up a connection with the master module and identifies itself to the master module. After exchange of apparatus identification it is established whether the master module is updated with respect to the internal data contained in the internal memory of the apparatus or not. If the master module is updated, the data is not transmitted once more. If, however, the master is not updated regarding the internal data of the apparatus, the data necessary to update the master is transmitted from the apparatus to the master module. After completion of the data transmission it is likewise established if the master module contains data relevant to the apparatus which is not present in the apparatus. If this is the case, the master module transmits the relevant data to the receiving means of the apparatus after which the data is stored in the internal memory of the apparatus. After storage of the received data or if no data transmission was necessary, the apparatus returns to its idle mode and the circle is completed.

FIG. 8 illustrates the general concept according to an embodiment of the invention with respect to communication. Here the system consists of the portable units: a master module, a doser, a BGM, the remote units: Remote Receiver, Physician/Expert Care-team and Stationary Unit and a Communication Interface between them.

The master module controls the information and data flow between itself and the other apparatuses and collects relevant data and information from all the other portable units. This data and information could e.g. be amount of medication, type of medication, body fluid concentration, time stamp (date and time) and inventory logistics. Additionally, the patient can manually input information and data related to amount of food, measurement of physical activity in the way described above. This data and information can then be transmitted via a communication interface (which may be built into the master module) to external units like a database for data acquisition of the patient's data over time or a computer which the patient uses to be kept informed about his treatment. Alternatively, all the apparatuses could communicate to all the others.

The information in the database can be accessed by a physician or an expert care-team who could easily and quickly check for compliance to e.g. a diet or treatment course/progress. The physician or expert care-team could send a notification (e.g. alert or warning) to the patient if the data shows an inappropriate future treatment span. The patient could also be notified of a future appointment in this way or receive guidance.

The system also makes it possible for the physician or expert care-team to give the patient a number of choices to a given situation. The patient could e.g. be informed that the blood glucose level/concentration is quite high and the patient could be presented with the choices of either exercising for given amount of time or administering a given amount of a given type of medication. The possibility of choices makes the patient feel more in control of the treatment and enhances the therapeutic value of the treatment. This could also be done automatically be the system.

Many of the above tasks could be fully automated by utilization of an expert system which is fully updated with the patient's data and condition and has access to the patient's behavior over time.

FIG. 9 illustrates two dosers and their communication paths The dosers are identical for the typical patient, one doser containing fast acting insulin, the other doser containing slow acting insulin. The dosers comprise a microcontroller and memory as shown in FIG. 5. The dosers are capable of holding information about the insulin type they contain. This information may either be obtained by the doser reading e.g. a bar code on the carpoule or the information may be input from the patient. Thus the features of the doser enable it to log information about the insulin treatment (insulin type size of the dose and time stamp)

One doser is equipped with a cap unit 73 which acts as a storage container for an extra insulin carpoule, needles etc. The storage container is capable of keeping track of the contents of the container which enables it to keep the inventory list updated, as described earlier in the present document.

The other doser is equipped with a cap unit 74 comprising a BGM, a microcontroller and memory. This enables the cap unit 74 to log information about the blood glucose concentration (with time stamp).

All the dosers 71,72 and the cap units 73, 74 comprise an interface which enables them to exchange data. In the present example the master device is the BGM cap unit 74, which, in addition to the local interface, comprises an interface that enables it to communicate with external units through standard communication links (RS-232, Wireless local area network, phone, cellular phone, pager, satellite link, etc.). Through these communication links, the patient's treatment data can be transferred to the patient's own computer 80 or via e.g. the telephone system 75 to the patient's electronic medical record on a central server 76. From here, the treatment data may be accessed by the patient e.g. from a web page, using a stationary computer 77, a laptop computer 78, a handheld computer 79, etc. Apart from the patient, the care team can access the patient's treatment data. The patient's master unit 74 can receive data from the central server 76, in addition to transmitting data.

This system has the advantage that the system can function on 3 levels:
1) If one of the patient's devices 71, 72, 73, 74 is isolated by means of communication, it will log data.
2) When the patient's devices 71, 72, 73, 74 are within communication distance, the treatment data are transferred to the master unit 74, enabling it to supply the patient with a overview of his treatment as well as warnings or alarms if data shows that a potential dangerous situation may occur.
3) When the master device 74 is connected to the central server 76 through standard communication links, the treatment data is transferred to the patient's electronic medical record. This enables an expert system on the central server to notify the care team if needed. The care team may send information back to the user or send help if needed.

Furthermore it is well known that due to the safety of the patient, the development of a medical device is a time consuming task. Using a local communication form between the patient's devices 71, 72, 73, 74 has the advantage that only the master device 74 need to be redesigned to keep up with the continuous change in the standard communication links.

1. A medical apparatus for use by a patient for assisting in the treatment of diabetes, wherein the treatment requires at least two separate actions be taken by the patient, the device comprising:
a. a first device for performing a first operation, the first device having a means for storing, processing, and/or displaying information;
b. a second device for performing a second operation, the second device having a means for storing, processing, and/or displaying information;
c. wherein the first and second devices are selected from the group consisting of:
i. a body fluid analyzer;
ii. a lancet device;
iii. a storage container; and
iv. a medication delivery device;
d. wherein the first or second device is a master module adapted for data communication between the devices and is configured to receive and store relevant information from itself and the remaining device; and
e. wherein the first and second device are detachably coupleable together by male/female coupling to form a single portable unit;
f. wherein each module contains a processor with programming to allow designation of a master module, such that should one of the first or second devices become unavailable, the remaining device is configured to be the master module, and when any two modules are configured together they are configured to automatically designate one as a master module.

2. The medical apparatus of claim 1, further comprising additional devices that are detachably coupleable to the master module via a lock mechanism so as to form a single portable unit.

3. The medical apparatus of claim 1, wherein each of the devices is, after being detached from the other, capable of operating independently.

4. The apparatus of claim 1, wherein the master module is configured to transmit data to an external unit.

5. The medical apparatus of claim 2, wherein each of the devices is, after being detached from each other is capable of operating independently.

6. The medical apparatus of claim 3, wherein the devices transmit and/or receive data from each other only after being coupled together.

7. The medical apparatus of claim 4, wherein the devices transmit and/or receive data from each other only after being coupled together.

8. A medical apparatus for assisting in a multi operation treatment of a disease comprising at least two separate operations, the apparatus comprising:
a. a first device for performing an operation;
b. a second device for performing an additional operation;
c. wherein the first and second devices are detachably coupled together to form a single portable unit and wherein the first and second devices may be decoupled and used apart from each other to perform their respective operations, wherein the coupling comprises inserting at least a portion of one device into at least a portion of the other device; and
d. wherein both the first or second device is capable of acting as a master module comprising a means for receiving data from the other device and for storing the data from itself and the other device.

9. A medical apparatus for assisting in a medical treatment that comprises at least two operations, the apparatus comprising:
a. a master module that performs an operation in the medical treatment;
b. a drug delivery module that performs an additional operation;
wherein the master module has an interior into which at least part of another module may be inserted thereby forming a single portable unit; and
wherein the master module is configured to receive information relating to the operation performed by the drug delivery module and is further configured to store information relating to the additional operation performed by the master module and to display both sets of information; and
wherein the drug delivery module contains a processor with programming to allow its designation as a master module such that the drug delivery module is configured as a master module if it is separated from the master module and combined with another medical apparatus.

10. The apparatus of claim 9, wherein the master module comprises a blood glucose monitor.

11. The apparatus of claim 9, wherein the master module is configured to transmit data to an external unit.

12. A system for assisting in the treatment of diabetes, the system comprising:
a first apparatus for performing a first function;
a second apparatus for performing a second function;
wherein either the first or second apparatus may be designated as a master module that is adapted to control data communications between the apparatuses and is adapted to receive and store relevant information generated in at least one of the apparatuses and wherein at least the two apparatuses are selected from the group consisting of:
a lancet device;
a blood glucose analyzer;
a drug administration device;
a sensor for sensing bodily parameters; and
wherein each apparatus contains a processor with programming to allow designation of a master module, such that should one of the apparatuses become unavailable one of the remaining apparatuses is configured to be the master module.

13. A system for assisting in the treatment of diabetes, the system comprising:
a first apparatus for performing a first function;
a second apparatus for performing a second function;
wherein either the first or second apparatus can act as a master module that is adapted to control data communications between the apparatuses and is adapted to receive and store relevant information generated in at least one of the apparatuses and wherein at least the two apparatuses are selected from the group consisting of:
a lancet device;
a blood glucose analyzer;
a drug administration device;
a sensor for sensing bodily parameters; and
wherein the master module is configured to send a message to a medical professional.
